(19) United States
(12) Patent Application Publication
(10) Pub. No.: US 2009/0035356 A1
(43) Pub. Date: Feb. 5, 2009

(54) MODIFIED BIODEGRADABLE POLYMERS, PREPARATION AND USE THEREOF FOR MAKING BIOMATERIALS AND DRESSINGS

(76) Inventors: Trung Bui-Khac, Montreal (CA); Ngoc Lang Ong, Montreal (CA)

Correspondence Address: CHOATE, HALL & STEWART LLP, TWO INTERNATIONAL PLACE, BOSTON, MA, 02110, US

(21) Appl. No.: 12/065,932

(22) PCT Filed: Sep. 5, 2006
(86) PCT No.: PCT/CA2006/001468

§ 371 Date: Aug. 15, 2008

(30) Foreign Application Priority Data
Sep. 6, 2005 (CA) 2,518,298

(51) Int. Cl. A61K 9/70; C07K 1/00; C07H 1/00; C07K 14/78; A61P 17/02; C08B 37/00; A61K 38/16; A61K 31/715

(52) U.S. Cl. 424/445; 530/402; 536/124; 530/353; 530/356; 536/123.1; 514/12; 514/54

(57) ABSTRACT

The invention concerns a method for preparing a modified biodegradable polymer in aqueous medium comprising at least two steps. The first step is a reaction between an amino acid, a peptide or a polypeptide and maleic anhydride to form a compound having an unsaturated vinyl-carboxylic acid function. In the second reaction step, the unsaturated diacid obtained in the first step is reacted with a biodegradable polymer having at least one primary amine function, such as a fibrous protein or a glycosaminoglycan. The preferred polymer used is collagen or chitosan. The invention also concerns the modified biodegradable polymer obtained by the method. The invention further concerns a biomaterial or a dressing containing the modified biodegradable polymer having biocompatible, cytocompatible, hemostatic, bactericidal and wound healing properties, and its medical, biomedical, pharmaceutical or cosmetic use.

### 1. FIELD OF THE INVENTION

The present invention concerns a new chemical process in aqueous medium for modifying biodegradable polymers. The process comprises a first reaction step in aqueous medium between an amino acid, a peptide or a polypeptide with maleic anhydride to form a vinyl-carboxylic acid which is, in a second step, reacted with a biodegradable polymer having at least a primary amine function such as a glycosaminoglycan, such as chitosan, or a fibrous protein, such as collagen or elastin.

The present invention also concerns modified biodegradable polymers obtained according to the process and their use in the medical, pharmaceutical and cosmetic fields, particularly for the manufacture of biomaterials and dressings having bio- or cyto-compatibility properties and hemostatic, bactericidal and/or wound healing properties.

### 2. DESCRIPTION OF THE PRIOR ART

### 2.1 Biomaterials

The field related to wound healing or surgical dressings for wound healing or surgery, to biomaterials or to fibrin adhesives, has been the subject of an intense development in the last century, and mainly with the objective of increasing the hemostatic effect of these various products in order to improve blood coagulation. To date the results of these developments have been the object of hundreds communications published in specialized reviews, newspapers or patents.

It is well-known by a Person of the art that a hemorrhage stops by itself through the natural process of blood coagulation. However, the consequences of a hemorrhage can vary according to its importance and origin. A small cut of the epidermis cannot be compared with a strong hemorrhage occurring during a surgical operation which leads to an important loss of blood and requires a blood transfusion.

The adhesive strength of a blood clot due to the presence of a polymerized fibrin network has been well known since 1909 when Bergel confirmed that fibrin can be used as physiological gluing substance with wound healing properties.

A few years later, this discovery allowed Grey (1915) to use fibrin plugs to induce coagulation of cerebral and hepatic hemorrhages. However, it was not until 1949 that Cronkite, then Tidrick and Warner, used fibrinogen combined with thrombin for cutaneous graft fixation.

Finally, thanks to important research by E. J. Cohn in 1946 on plasma protein fractionation, proteins involved in coagulation began to be exploited and a few years later, the mechanism by which various proteins are involved in coagulation was understood.

In spite of the important progress in the field of wound healing by the use of biological substances, the sixties saw the emergence on the market of tissue adhesive-containing synthetic products such as a very powerful adhesive of the cyanoacrylate family, polymerized in a few seconds. However, its use caused important cellular toxicity. Other synthetic adhesives of the same family then appeared, but with longer side chain radicals having hemostatic, bacteriostatic and wound healing properties. However, these adhesives also caused inflammatory reactions and tissue toxicity that was still too important.

In 1967, formaldehyde-based adhesives containing gelatin, resorcin and formalin were introduced. They provided a certain improvement with regard to toxicity, but unfortunately also provoked allergic reactions and tissue toxicity related to the presence of formalin. Inflammatory reactions, tissue toxicity, and allergies caused the rejection of these adhesives that were poorly biocompatible.

Ashton et al. reported in U.S. Pat. No. 3,364,200 (1968) that conventional hemostatic gauze pads or similar articles filled with a hemostatic material such as ferric chloride, thrombin, or equivalents thereof, have been used for many years to stop bleeding. However, these surgical hemostatic materials are criticized because they cannot be left in situ in a closed wound because of the risk of neighboring tissue reacting with foreign bodies. Moreover, if these materials are left inside the healed wound, the wound would have to be re-opened which would disrupt the blood clot which has formed, thereby causing renewed bleeding. Ashton et al. therefore mention that there exists a vital need for hemostatic tissue that could remain in a closed wound without however causing serious reactions in neighboring tissues. In light of this, Ashton et al. propose the use of oxidized cellulose that not only has hemostatic properties, but that is also absorbable by animal tissue. Ashton et al. thus provide hemostatic oxidized cellulose materials having improved stability against deterioration during storage. The oxidized cellulose is derived from wood pulp, cotton, cotton, ramie, linters, jute, paper or similar materials and regenerated cellulose or rayon produced by either the viscose or Bemberg process. This invention has led to the marketing of Surgicell™ by Johnson & Johnson.

The use of the adhesive properties of fibrin via a very strong concentration of fibrinogen and Factor XIII with a fibrinolysis inhibitor to carry out an experimental joining of nerves was developed in 1972 by H. Matras. Following his work, the first biological adhesive elaborated and manufactured by Immuno AG (Vienna, Austria) appeared on the European market in 1978. This adhesive is used to stop hemorrhage and consists of human coagulation proteins, mainly containing fibrinogen and Factor XIII. This composition is mixed right before its use with an antifibrinolytic solution of bovine origin: Aprotinin. This solution is mixed progressively with a thrombin solution of bovine origin for application on wounds. Since then, several other similar products are commercially available, particularly in surgery.

In parallel with the development of biological adhesives, substances having natural or chemically transformed hemostatic properties were introduced on the market. These new products are also called “biological dressings” and among the other substances used, polysaccharides bearing only glucosic units such as cellulose and its cellulose derivatives, gums and alginates extracted from algae are found. Polysaccharides including one or more amino groups or a repetitive unit with an amine function are called aminopolysaccharides or glycosaminoglycans.

Glycosaminoglycans are thus long non-branched polysaccharide chains made by the repetition of the same disaccharide unit. Disaccharides of this unit comprise a monosaccharide carrying a carboxylic group named galacturonic acid and a second saccharide carrying a N-acetylamine group named acetylglucosamine or N-acetylgalactosamine. These glycosaminoglycans are present in abundance in connective tissue, in particular in bone and cartilaginous tissues. The glycosaminoglycans most often used are, for example, hyaluronic acid, chondroitin sulphate, dermatane, heparane sulphate or heparin. These same connective tissues also contain fibrous proteins of collagenous structure and elastin, these two proteins also having very interesting medical applications in the field of wound healing.

Other types of glycosaminoglycans are present in invertebrate shells or such as Chitin [β-(1,4)-2-acetamido-2-diseoxy-D-glucose or poly N-acety-D-glycosamine]. The deacetylation of chitin leads to the formation of Chitosan [β-(1,4)-2-amino-2-desoxy-D-glucose]. Chitin is the second most abundant natural polysaccharide after cellulose.

It is well-known that glycosaminoglycans and fibrous proteins have naturally biocompatible, biodegradable, hemostatic and wound healing properties. These characteristics allow them to be classified as biopolymers or biomaterials. Many medical or esthetic applications have been developed with these biomaterials, especially in the field of wound healing. Each native biopolymer has its own biological and physico-chemical properties, some have more fragile biomechanical properties or are degraded in vivo more quickly than others. According to the desired use, a lot of work on the chemical structural modification of these biopolymers has been performed to obtain a biomaterial that is mechanically and chemically more robust, more absorbent or biochemically more active. Certain modifications lead to the modification of volume or surface properties of the biopolymers so that these new biomaterials provide an exponential absorption of their thickness when they are in contact with the aqueous medium.

K. Park et al. (Biodegradable Hydrogels for Drug Delivery-Technomic, Publishing Co., Inc. 1993, page 107) have classified polysaccharides according to following sources:
i. Algae: Agar-agar, furcelleran, alginate, carrageenan;
ii. Vegetable: plant extracts (starch, pectin, cellulose), gum exudates (arabic gum, tragacanth, karaya, ghatti) and seed gum (guar gum, carob seed gum);
iii. Microbial: xanthane, pullulane, scleroglucane, curdlane, dextrane, gellane;
iv. Animal: chitin and chitosan, Chondroitin Sulphate, Dermatane Sulphate, heparin, keratane, hyaluronic acid.

Synthetic polymers such as polyols and their derivatives, poly(vinylalcohols), polyvinylpyrrolidones, polyesters, or the polyanhydrides are very well exploited for various pharmaceutical and medical applications. These polymers, considered as biodegradable materials, are often called “Hydrogels”.

The biomaterials cited above can only be carried out and used by taking into account their hemostatic properties or by incorporating them with coagulation proteins such as fibrinogen and thrombin, and proteins that support wound healing. Pharmaceutically acceptable ingredients can also be added like antibiotics, antibacterial agents, anti-cancer agents, etc.

### 2.2 Modification of Biomaterials

As mentioned hereinabove, the first step of the process of biodegradable polymer modification according to the invention consists of a reaction between maleic anhydride and an amino acid or one of its derivatives.

### 2.2.1. Reaction Between an Amino Acid or Derivatives Thereof and Maleic Anhydride

The reaction between maleic anhydride and an amino acid has been the subject matter of several patents or scientific articles.

Japanese patent JP56012351 (Uesugi Hideyuki et al., 1981) discloses the obtaining of N-acylaminoacid by the reaction of a maleic or succinic anhydride with an amino acid in the presence of an inert organic solvent such as tetrahydrofuran (THF) or dioxane, leading for example, to N-β-carboxypropionyl-DL-α-alanine. The reaction is carried out at a temperature ranging between 40 and 110° C., and is used as an intermediate in the synthesis of organic compositions such as surfactants or in the extension of chains to high molecular weight such as polyamides or polyesters.

Japanese patent JP 59197459 (Itou Fumisaku et al., 1984) discloses a method of synthesizing obtaining materials comprising polyamides resistant to impact and fatigue due to the flexibility of maleic anhydride grafted on an elastomer which is then reacted with an amino acid.

U.S. Pat. No. 5,665,693 (Kroner et al., 1997) discloses a method of preparing detergents or cleansers having a very low rate of phosphate or an absence of phosphate. This method consists in reacting maleic anhydride, maleic acid and/or fumaric acid on proteins or hydrolyzed protein which did not extend beyond the dipeptide stage. The reaction took place at a temperature ranging between 120 to 300° C. under high pressure and in a mixture of aqueous and organic solvents.

Japanese patent JP 2000319240 (Imai Masaru et al., 2000) discloses a method of producing a maleinamic acid by reacting maleic anhydride on an amino carboxylic acid in the presence of an ammonium salt and in a non-polar hydrocarbon solvent.

Butlet P. J. G. et al. (Biochemical Journal (1967), vol. 108, p. 78-79; (1969), vol. 112, p. 679-689) showed that maleic anhydride reacts quickly and specifically with amino groups of proteins and peptides with formation of maleyl-proteins. The maleyl-amino group is very stable under neutral or alkaline pH conditions, but is easily hydrolysed under acidic pH conditions. This characteristic allows the blocking of amino groups in a reversible way. The maleyl group could be removed because of the protonic forms of the free carboxylic groups which catalyze the hydrolysis of the amide bonds.

Dixon H. B. F. et al. (Biochemical Journal, (1968), vol. 109, p. 312-314) also highlighted the reversible blocking of the amino groups of proteins, by the use of citraconic anhydride (or anhydride 2-methylmaleic) or anhydride 2,3-dimethylmaleic instead of maleic anhydride.

Riley M. et al. (Biochemical journal, (1970), vol. 118, p. 733-739) also developed the reversible reaction of amino groups of proteins with exo-cis-3,6-endoxoo-Δ-tetrahydrophthalic anhydride.

De Wet P. J. (Agroanimalia, (1975), 7 (4), p. 101-104), described the synthesis of maleylmethionine by reacting maleic anhydride with L-methionine. According to the author, this compound is stable at pH 5.0 and is hydrolyzed to 60% at pH 2.20 after 8 hours. This reversibility of the reaction of maleic anhydride with an α-amino acid is proposed as a possible method for the protection against deamination in the rumen.

However, none of the documents mentioned hereinabove describes the synthesis of a maleyl-amino acid compound directly by reacting a maleic anhydride with an amino acid in an aqueous medium.

### 2.2.2 Modification of a Biodegradable Polymer

As aforesaid, the second step of the process according to the invention consists in the modification of a biodegradable polymer and, in particular, collagen or chitosan.

Fibrous proteins, such collagen, and glycosaminoglycans, such as chitosan, are often transformed or polymerized to obtain products with new features, corresponding to new required or expected uses. These products have very many applications in the medical, pharmaceutical, aesthetic or cosmetic fields.

2.2.2.1. Collagen

Collagen is a fibrous glycoprotein present in connective and interstitial tissue. Having a high molecular weight structure, they are a very important element of the extracellular matrix of the human body. There are various types of collagen according to their localization with properties which allow the collagen to be classified as one of the principal essential elements of skin, tendons, cartilage and bone. Collagen is inextensible and resists well to traction. It is particularly essential to the process of wound healing

From its hemostatic and wound healing properties, collagen finds many interesting applications in the biomedical field. The following are some major applications of collagen:
Surgical products (topical hemostats and suture wires),
Implants (orthopedic, dental or bladder implants),
Injectable cosmetic products (facial wrinkles and small wrinkles, scars),
Ophthalmology (corneal screen, contact lenses, viscoelastic solution),
Skin substitute.

Extraction, purification and transformation of collagen allow for the obtaining of an absorbable medical biomaterial. As the need and necessity to adapt the product to the sites used increase the natural state of collagen has been modified either by chemical or physical polymerization (heat, radiation, grafting, mixture with other polymers) so that the manufactured collagen-based are products more effective and serve the needs of clinicians and patients and ensures comfort to the patient as safer use of the product (non-toxic, non-immunogenic and non-inflammatory), such as effectiveness, biocompatibility, bioresorbability, tolerance, elasticity or workability.

One of the first applications of a collagen-based product was the control of excessive bleeding. The oldest product which was prepared for that purpose was Gelfoam™. U.S. Pat. No. 2,465,357 (Correll, 1949) indeed describes a gelatin sponge which is permeable to liquids and is water-insoluble, having the physical characteristics of a sponge while being absorbable by animal bodies. The sponge according to the invention is a porous substance which must be relatively soft when it is wet and must present several fine interstices to place a certain quantity of therapeutic agent within it and to allow a slow release of this agent. The sponge also acts like an effective material to absorb the free flow of fluids like blood and exudates around a wound. Surgeons have used this hemostatic product since 1940, but it has been proven to be inefficient. Gelatin only plays a passive role in topical hemostasis where bleeding is mechanically controlled by pressure exerted on the wound or the cut. The passive hemostatic effect of the gelatin is useful only for a restricted use where the need for a gelatin sponge allowing the absorption of abundant blood is necessary. These sponges are freeze-dried products and manufactured from a purified gelatin solution which thus undergone a special treatment. Placed on the wound, they allow the absorption of a quantity of body liquid equivalent to several times their weight.

Because of the low efficiency of gelatin sponges, research was quickly directed towards the isolation of collagen, its purification and its transformation to make a more performant product. A topical collagen-based dressing appeared on the market around 1980. The development of this material continued with other applications like corneal protection followed by injectable collagen for the cosmetic treatment of wrinkles and scars. Patents directed to the isolation, transformation, application and methods of manufacture of these products with collagen are innumerable, and only a few patents relating to polymerization or grafting of collagen alone or mixed with glycosaminoglycan in a chemical way are cited herein. In general, the authors wish to obtain an absorbable and effective biomaterial having numerous uses such as:
an active dressing to help stop hemorrhage during surgery,
treatment of dental wounds,
wound closing,
treatment of burns,
treatment of incontinences,
eye protection after abrasion or cataract surgery,
a skin graft substitute,
a bones graft substitute,
elimination of wrinkles, acne and scars, or
prevention of post-surgical adhesions.

Native collagen cannot be used for all the applications mentioned hereinabove. The most common reason for this is its fast degradation in vivo, its lack of elasticity which prevents it from adapting to the contour of wounds and its weak force of traction. This is why the authors cited hereinafter have sought to modify the natural state of collagen to find new applications for it. Collagen modifications are often related to polymerization or grafting of other groups or other polymers of natural or chemical origin. In general, the change in the initial structure of collagen considerably reduces the immunological reaction.

Various collagens of different types and grades (with and without telopeptides) are commercially available. These collagens are of animal or human origin. By nature, collagen has biochemical and physicochemical characteristics which are relatively well adapted for its use as a biomaterial. In particular, these characteristics are good biocompatibility, as well as exceptional biodegradation and hemostatic properties. On the other hand, collagen has a weak traction force. Products containing collagen, like medical, surgical or cosmetic implants, encountered problems such as difficult manual handling. Since collagen is not easily foldable, it is difficult to follow the contour of a wound. Moreover, the biodegradation of collagen for certain applications is considered as being too fast, for example, a long period of time is required for an implant to gain palliative and curative action. To answer these expectations of the patient, medical collagen-based products must be chemically modified and often by coupling collagen with another chemical group.

Since it is known that the major component of the skin is collagen, a logical approach for the development of a skin substitute has led to studying the behavior of reconstituted collagen, placed in contact with living tissues.

This approach has been explored by a great number of researchers using a general procedure of extraction and purification to various degrees of animal collagen. This purified collagen was then converted into film or other structures for use such as hemostatic dressings or implants in a living tissue in order to determine their behavior in vivo. In fact, collagen purification consists of digesting the non-helical portion of collagen, often called “telopeptide”, by proteolytic enzymes in order to noticeably reduce immunoreactivity.

Enzymatically modified collagen was prepared and tested by Rubin and Stenzel [Rubin, A. L. and Stenzel, K. H., in Biomaterials (Stark, L. and Aggarwal, G., Eds.), Plenum Press, N.Y. (1969)] who showed that the treatment does not cause any immunoreaction compared to non-modified collagen. This difference in behavior would be explained in that the enzyme used efficiently removes the telopeptides from collagen without destroying the initial molecular structure.

U.S. Pat. No. 3,742,955, Battista et al. reported that prepared or treated collagen is useful in surgery for wound treatment, and E. Peacock, Jr. et al. in Ann. Surg. 161, 238-47, February, 1965, reported that collagen has hemostatic properties when used as a wound dressing. Battista et al. further reported that fibrous collagen and fibrous products derived from collagen when properly prepared and when wet with blood will not only provoke hemostasis, but will also demonstrate completely unexpected adhesiveness to severed biological surfaces in warm-blooded animals. Battista et al. also provided a method of preparing finely divided collagen fibres and fibrous products derived from collagen which are useful hemostatic agents having unique adhesive properties. This invention has led to the marketing of Avitene® by ACECON.

U.S. Pat. No. 4,488,911 (Luck et al., 1984) describes a method for preparing collagen in solution (CIS), wherein native collagen is extracted from animal tissue and diluted in aqueous acid, followed by enzymatic digestion with pepsin, trypsin, or Pronase™. The enzymatic digestion removes the telopeptide portions of the collagen molecules, and to isolate “atelopeptide” collagen in solution. The atelopeptide collagen in solution so produced is substantially non-immunogenic, and is also substantially non-crosslinked due to loss of the primary crosslinking regions. The collagen in solution may then be precipitated by dialysis in a moderate cutting environment to produce collagen fibers which resemble native collagen fibers. The precipitated, reconstituted fibers may additionally be crosslinked using a chemical agent (for example, aldehydes such as formaldehyde and glutaraldehyde), heat, or radiation. The resulting products are suitable for use in medical implants due to their biocompatibility and reduced immunogenicity.

Despite the fact that the protein structure remains unchanged after its purification, purified collagen or atelopeptide collagen is more quickly degraded in vivo when implanted in mammals. For this reason, polymerized atelopeptide collagen was cross-linked or grafted using chemical radicals to increase the fibrous network and to allow the modified product to resist longer to its own biodegradation in vivo and to increase its traction force or viscosity in solution.

Many studies were undertaken to develop the possibilities of collagen coupling thanks to the presence and availability of many amino groups (NH₂) on its molecular structure. Among the new inventions, four main coupling groups for this protein are found.

### Group 1:

The first group uses a reticulating or polymerizing agent which forms a bridge between the same molecules or, via this bridge, other molecules could be grafted onto this bridge.

The reagents most frequently used are mono or dialdehydes leading, for example, to the formation of a methylene bridge with formaldehyde or to the formation of an imine and an aldol with glutaraldehyde, a bifunctional crosslinking agent, which reacts with free amines. The major problem produced by these formations is due to the presence of final aldehyde group (—CHO) which, once this group salted out, will be transformed into a cytotoxic and irritant dialdehyde polymer.

U.S. Pat. No. 2,900,644 (Rosenberg et al. 1959) discloses a method for the preparation of a tubular implant made from an ox carotid artery whose solidity is reinforced by treatment with formaldehyde. However, the use of collagen polymerized by an aldehyde involves inflammatory reactions.

To reduce the inflammatory reactions, Grillo et al. (J. Surg. Abstr., (1961), vol. 2, p. 69) suggested controlled polymerization of collagen by formaldehyde to slow down its resorption speed. Grillo et al. also showed that the immune response due to the reconstituted collagen implants was minimal.

In U.S. Pat. No. 3,093,439 (Bothwell et al. 1963), the authors used a dialdehyde starch obtained by the oxidation of starch with iodate for treating ox carotid arteries, which are used as implants.

U.S. Pat. No. 3,157,524 (Artandi), discloses methods for preparing collagen sponges by using formaldehyde. With the same crosslinking agent, the inventor also obtained porous collagen tubes.

U.S. Pat. No. 3,823,212 (Chvapil et al., 1974) discloses a method to obtain membranes or sponges containing reticulated collagen by treating collagen with glutaraldehyde at low temperature, from −5 to −40° C. and over a period of time from 1 day to 30 days. Antibiotics can be added to collagen for its preparation. The products obtained had medical applications such as for the treatment of skin burns or abrasions (where broadly affected surfaces must be covered to avoid wound infection), to help wound healing by getting active therapeutic compounds, or to prevent wound maceration and slow absorption of exudations.

U.S. Pat. No. 4,060,081 (Yannas et al., 1977) discloses the use of collagen and mucopolysaccharides as synthetic skin. Such material is crosslinked using glutaraldehyde, a bifunctional crosslinking agent, which reacts with free amines.

Another use of glutardialdehyde to reticulate collagen was described in U.S. Pat. No. 4,131,650 (Braumer et al., 1978). The authors described a skin treatment wherein an aqueous paste is applied to the skin, left in contact therewith for a period of time and thereafter removed, in which the improvement comprises placing a foil over the paste, the foil containing at least about 3 percent of water-soluble collagen by weight and having a water permeability of more than about 0.1 gram/dm²2 minute, whereby collagen is transported through the paste and is absorbed by the skin. Preferably, the foil is about 0.01 to 0.03 mm thick has a cross-linking rate corresponding to that produced by about 0.1-0.5 percent by weight of glutardialdehyde applied in an acid medium. The foil may further contain a cosmetically active agent such as an amino acid, peptide, protein, hormone, placenta-extract, phosphatide, tissue-extract, fresh cells and vitamins. The paste may be dried by heating, producing shrinkage of the foil to increase contact with the skin.

Another enzyme-conjugated (alkaline phosphatase) by gluteraldehyde collagen polymerization reaction was described in U.S. Pat. No. 4,409,332 (Jefferies et al., 1983). The authors obtained a gel containing polymerized collagen without an inflammatory reaction used to produce implantable membranes, stitch wire, an injectable and biocompatible gel, a drug transporting support, an injectable collagen used as an implant and a method for increasing soft tissue.

U.S. Pat. No. 4,424,208 (Wallace et al., 1984) describes an improved collagen formulation suitable for use in soft tissue augmentation. Wallace's formulation comprises reconstituted fibrillar atelopeptide collagen (for example, Zyderm® collagen) in combination with particulate, crosslinked atelopeptide collagen dispersed in an aqueous medium. The addition of particulate crosslinked collagen improves the implant's persistence, or its ability to resist shrinkage following implantation. The crosslinked reagent used is glutaraldehyde.

U.S. Pat. No. 4,582,640 (Smestad et al., 1986) discloses a glutaraldehyde crosslinked atelopeptide CIS preparation (GAX) suitable for use in medical implants. The collagen is crosslinked under conditions favoring intrafiber bonding rather than interfiber bonding, and provides a product with higher persistence than non-cross-linked atelopeptide collagen, and is commercially available from Collagen Corporation under the trademark Zyplast® implant.

U.S. Pat. No. 4,597,762 (Walter et al., 1986) discloses a method of preparation of type I of collagen and its polymerization by glutaric dialdehyde in presence of ficin and L-cysteine. The obtained product is used in human and veterinary medicine.

U.S. Pat. No. 4,600,533; U.S. Pat. No. 4,655,980; U.S. Pat. No. 4,689,399 and U.S. Pat. No. 4,725,671 (Chu et al.) discloses obtaining collagen membranes with desired properties by using a variety of gel-forming techniques in combination with methods for converting the gels to solid forms. The properties of these membranes or other solid forms may be further altered by cross-linking the collagen preparation either after formation of the membrane or gel, or most preferably by mixing cross-linked collagen with solubilized collagen in the original mixture used to create the gel. The cross-linked reagents are formaldehyde, glutaraldehyde, glyoxal and so forth. The obtained collagen membranes are for medical use.

U.S. Pat. No. 4,980,403 (Bateman et al.); U.S. Pat. No. 5,374,539 (Nimni et al.) and U.S. Pat. No. 5,411,887 (Sjölander) disclose the use of a crosslinking agent, like glutaraldehyde, to polymerize collagen, which is used for manufacturing products of human and veterinary medical applications, such as bioprosthetics, or gel and films for wound treatment.

A major disadvantage of the use of crosslinked collagen is the negative biological reaction due to the salting out of aldehyde, a reagent often used for polymerizing collagen and making it insoluble for several applications. The detachment of aldehyde linked to the crosslinked collagen shows cell cytotoxicity, specifically for fibroblasts (Speer et al., J. Biomedical Materials Research 1980,14, p. 753; Cook et al., British J. Exp. Path. 1983, 64, p. 172). Recent evidence suggests that glutaraldehyde polymers, unlike the manomeric form of glutaraldehyde, form reticular bonds between collagen molecules; these bonds can be rearranged for releasing glutaraldehyde and glutaraldehyde polymers (Cheung, D. T. and Nimni, M. D., Connective Tissue Research 10,187-217,1982).

### Group 2:

Collagen coupling agents used to avoid secondary and parasite reactions due to free aldehydes are the second group of coupling agents. Among these new coupling agents, the following are cited:
acylation reaction via an anhydride (dicarboxylic compounds) producing an amine or ester bond, the anhydrides most often used being succinic, maleic, glutaric, benzoic, lauric, diglycolic, methylsuccinic or glutaric methyl anhydride;
sulphonation and phosphorylation reactions by phosphorylated and sulphonated halogens which create intra and intercatenary bonds;
the use of carbodiimides, diisocyanates or diamines allowing to create amide bonds; —formation of a disulphide bridge —S—S—; or
obtaining an aldehyde function with polysaccharides, which comes to be added on the collagen to extend the polymer network.

U.S. Pat. No. 4,404,970 (Sawyer) describes the modification of by reacting its acid functions with amines in the presence of EDC (1-ethyl-3,3-dimethylaminopropyl) carbodiimide.

U.S. Pat. No. 4,703,108 and U.S. Pat. No. 4,970,298 (Silver et al.) teach the preparation of a matrix, a sponge or a film containing collagen by contacting collagen with a crosslinking agent chosen among a carbodiimide and an active ester derived from N-hydroxysuccinimide followed by a strong dehydration.

U.S. Pat. No. 4,713,446 (DeVore et al 1987) discloses a chemically-modified collagen prepared by reacting native collagen with di or tri-carboxylic acid derivatives such as halides, sulfonyls, anhydrides, or esters as coupling agents. The reaction is controlled in order to limit the degree of cross-linking. The reaction takes place at the level of the residual amine functions of the lysine found on the collagen. The obtained product is dissolved in a physiological buffer providing a viscoelastic solution having therapeutic applications in a variety of surgical procedures, particularly in ophthalmology. The crosslinking reagents are, in particular, succinic anhydride and succinyl chloride; phthalic anhydride; or glutaric anhydride.

U.S. Pat. No. 4,837,285 (Berg and al., 1989) discloses the crosslinking of collagen matrix beads by dispersing the beads in a carbodiimide solution. The crosslinking may be used in combination with severe dehydration at temperatures between 50° C. and 200° C. in a vacuum of less than 50 torr for 2 to 92 hours.

U.S. Pat. No. 4,958,008 (Petite et al., 1990) disclose a chemical process ensuring the blocking of the acid side groups present on collagen and thereby avoiding the use of glutaraldehyde which could lead to the phenomena of toxicity and calcification. According to the invention, the process of crosslinking the collagen by introducing azide groups essentially comprise the following steps: esterification of the free acid groups of the collagen, transformation of the esterified groups into hydrazide groups followed by transformation of the hydrazide groups into azide groups by the action of nitrous acid, and characterized in that each step is separated by a washing with an aqueous salt solution.

U.S. Pat. No. 5,412,076 (Gagnieu C., 1995) discloses a crosslinked modified collagen which is soluble in water and/or in aprotic polar organic solvents, wherein the free thiol groups belonging to residues of cysteine or analogs thereof are crosslinked by the formation of disulfide bridges, and such, to yield gels or crosslinked products in the presence of mild oxidizing agents, affording excellent control over the kinetics and the degree of crosslinking. The diverse applications in the field of are adhesives, biomaterials for prostheses, implants, or other medical articles.

U.S. Pat. No. 5,332,802 (Kelman et al., 1994) describes the production of a chemically modified, crosslinked, telopeptide from a human donor, for implanting into the same donor. The chemical modification of the tissue is performed by acylation and/or esterification, to form an autoimplantable, collagenin in the form of a solution.

U.S. Pat. No. 5,874,537 (Kelman et al., 1999) describes collagen-based compositions as adhesives and sealants for medical uses. Prior to polymerization, soluble or partially fibrillar collagen monomers in solution are chemically modified with an acylating agent, sulfonating agent or a combination of these two. Accordingly, the collagen compositions prepared can be used as medical adhesives for bonding soft tissues or be made into a sealant film for a variety of medical uses such as wound closures or tendon wraps for preventing adhesion formation following surgery.

U.S. Pat. No. 5,866,165 and U.S. Pat. No. 5,972,385 (Liu et al., 1999) disclose the preparation and the use of a support matrix to allow the growth of tissues, such as bone, cartilage or soft tissue. A polysaccharide is reacted with an oxidizing agent to open sugar rings on the polysaccharide to form aldehyde groups. The aldehyde groups are reacted to form covalent bonds to collagen. Preferentially, the polysaccharide used is hyaluronic acid.

U.S. Pat. No. 6,165,488 (Tardy et al., 2000) discloses a similar process using a polyaldehyde macromolecule of natural origin obtained by the crosslinking of collagen with periodate for uses such as biocompatible, bioresorbable and non toxic materials for surgical or therapeutic use.

U.S. Pat. No. 6,309,670 (Heidaran et al., 2001) discloses a method of treatment for bone tumors comprising the administration of a matrix comprising collagen, a polysaccharide and a differentiation factor. A polysaccharide is reacted with an oxidizing agent to open sugar rings on the polysaccharide to form aldehyde groups. The aldehyde groups are reacted to form covalent bonds to collagen. The polysaccharides used are hyaluronic acid dextran or dextran sulphate-polyaldehyde.

U.S. Pat. No. 6,127,143 (Gunasekaran, 2000) discloses the use of a phosphorylation method to obtain a biocompatible product from purified collagen which is produced by using two proteolytic enzyme treatments and a reducing agent. Prior to phosphorylation, the purified collagen is delipidated, and then treated by compressing, dehydrating, dispersing and drying to form collagen fibers. The purified and biocompatible collagen may be used in transplantation or hemostasis, and may be combined to compounds such as antimicrobials, antivirals, growth factors and other compounds suitable for biomedical uses.

U.S. Pat. Nos. 5,492,135; 5,631,243 and 6,448,378 (DeVore et al., 1996, 1997, 2002) describe the preparation of a collagen film which is rapidly dissolved at 35° C. and used as a vehicle for delivering a dose of therapeutic compound to a specific tissue site. This collagen film is made from telopeptide poor in collagen which is modified with glutaric anhydride.

U.S. Pat. No. 6,335,007 (Shimizu et al., 2002) discloses a collagen gel which is obtained by crosslinking a polyanion and a carbodiimide. Such polyanions used are alginic acid, Arabic gum, polyglutamic acid, polyacrylic acid, polyaspartic acid, polymalic acid, carboxymethylcellulose and carboxylated starch and the water soluble carbodiimides are 1-ethyl-3-(3-dimethylaminopropyl) carbodiimide hydrochloride. The product claimed consists of a kit for producing a collagen gel comprising a collagen aqueous solution, a polyanion aqueous solution and a carbodiimide aqueous solution. The kit is used as an adhesive directly on the human body with hemostyptic, obstruent or dead space fillers for the fabrication of artificial blood vessels, artificial tubes or artificial esophaguses.

U.S. Pat. Nos. 6,969,400 and 6,911, 496 (Rhee et al., 2005) describe a crosslinked polymer composition that includes a first synthetic polymer containing multiple nucleophilic groups covalently bound to a second synthetic polymer containing multiple electrophilic groups. The first synthetic polymer is preferably a synthetic polypeptide or a polyethylene glycol that has been modified to contain multiple nucleophilic groups, such as primary amino or thiol groups. The second synthetic polymer may be a hydrophilic or hydrophobic synthetic polymer, which contains or has been modified to contain two or more electrophilic groups, such as succinimidyl groups. The compositions may further include other components, such as naturally occurring polysaccharides or proteins (such as glycosaminoglycans or collagen) and/or biologically active agents. This patent also teaches methods for using crosslinked polymer compositions to improve adhesion between a first surface and a second surface; to increase tissues to prevent the formation of surgical adhesions and to coat a surface of a synthetic implant.

U.S. Pat. No. 6,962,979 (Rhee et al., 2005) discloses novel crosslinked biomaterial compositions which are prepared using hydrophobic polymers and a crosslinking agent. Hydrophobic polymers used are mainly those that contain two or more reactive succinimidyl groups, including disuccinimidyl suberate, bis(sulfosuccinimidyl) suberate, and dithiobis(succinimidylpropionate). The crosslinked biomaterial compositions prepared using mixtures of hydrophobic and hydrophilic crosslinking agents are also disclosed. The compositions can be used to prepare implants for use in a variety of medical applications.

U.S. Pat. No. 6,916,909 (Nicolas et al. 2005) describes novel collagen peptides that are modified by grafting free or substituted thiol functions carried by mercaptoamine radicals. The essence of this invention is to provide thiol collagens that can be cross-linked in a sufficient and controlled manner by forming sulfate bridges and which are also biocompatible.

U.S. Pat. No. 6,790,438 (Constancis et al., 2004) discloses a modified collagen peptide for preventing post-operative adhesions. Here again, the collagen peptide is modified by grafting thiol functions provided by mercaptoamine radicals that are exclusively grafted on the aspartic and glutamic acids of the collagen chains by means of amide bonds.

### Group 3:

The third group relates to copolymerization reactions between collagen and a polymer by a covalent bond by providing more or less a crossed conformation. The polymers most associated with collagen are acrylic derivatives, acrylonitriles, styrenes, polyurethanes, polyalcohols and silicones.

U.S. Pat. No. 4,452,925 (Kuzma et al. 1984) discloses hydrogels prepared by polymerizing a mixture containing an important quantity of organic monomer such as N,N-dimethylacrylamide, 2-hydroxyethylmethacrylate, dimethylaminoethylmethacrylate or methoxytriethylene glycol methacrylate, and a minor amount of solubilized collagen. The reactants used are at least partially soluble in the aqueous reaction medium. The hydrogels thus prepared are novel, shaped articles having utility in the medical and cosmetic fields.

### Group 4:

The fourth group of collagen coupling agents consists in making a dense network by the creation of covalent bonds only between collagen molecules without the incorporation of other groups of molecules. This group includes:
irradiation by ultraviolet, beta or gamma rays producing a deamination and thus allowing a coupling of imine and aldol bonds or irradiation by free radicals released by these sources of rays, which form structures with the covalent bridges. This method of coupling can be carried out only with a weak energy source; the use of a high energy source leads to hydrolysis or denaturation of collagen;
dehydration under pressure and at high temperature (≧100° C.) leads to the formation of an ester amide bond of both intra and intermolecules of lysinoalanine. Carbodiimides, such as cyanamide and dicyclohexylcarbodiimide, are used as reagents in this process;
oxydo-reduction producing a deamination by oxidation of the terminal of the amino groups with the formation of an aldehyde group. This method often uses cations (Cu²⁺, Fe²⁺, Al³⁺) in the presence of sulphites or nitrites and is very widely used in the tannery of leather; and
functional activation of carboxyls producing azide acids having a very selective reactivity to the amino functions (—NH₂) which lead to the formation of an amide bond.

U.S. Pat. No. 4,614,794 (Easton et al., 1986) discloses the obtaining of a biomaterial from a mixture of collagen and sodium alginate. After a freeze-drying step, these compounds are transformed into a coupled complex, i.e. a dehydrothermal complex, by heating at 115° C. under a vacuum, for 48 hours.

U.S. Pat. No. 5,331,092 (Huc et al., 1994) discloses a method of crosslinking or coupling between collagen molecules by heating the lypholized-collagen product at 110° C. under a vacuum of 400 microbars for 10 hours.

U.S. Pat. No. 4,931,546 (Tardy et al., 1990) discloses the coupling between the collagen molecules, at neutral or basic pH, thanks to aldehyde functions formed by action of a periodic acid or sodium salts thereof with the collagen molecule.

U.S. Pat. No. 4,958,008 (Petite et al., 1990) discloses a method of obtaining a biomaterial for the preparation of a bioprothesis containing collagen which was coupled thanks to the formation of an azide-collagen group and the amino of the lysine terminal of the collagen by providing an amide bond. This formation gives an internal coupling reaction of the collagen network.

2.2.2.2. Chitosan

Chitosan results from desacetylated chitin and is a polysaccharide including the random distribution of D-glucosamine bound by β-(1-4) (desacetylated unit) and N-acetyl-D-glucosamine (acetylated unit). From its hemostatic property and affinity with lipids, chitosan has become for more than two decades a largely exploited substance for its applications in the medical, pharmaceutical, cosmetic and dietetic fields. Medical applications are also based on chitosan characteristics such as its biocompatibility (minimizing the inflammatory reactions), its bioresorbability and biodeterioration. Moreover, the chitosan is well-known as being a good substrate for cellular colonization stimulating cell growth and thus increasing the healing rate of open wounds by stimulating the immune response and tissue rebuilding by preventing microbial infections and by absorbing exudates. Indeed, chitosan also has antibacterial and antimicrobial properties.

U.S. Pat. No. 4,031,025 (Vanlerberghe et al., 1977) discloses the method of acylation of chitosan with a saturated or unsaturated organic diacid anhydride. The resulting product is used as a skin moisturizer in a cosmetic agent composition. The saturated anhydrides used are succinic anhydride, acetoxysuccinic anhydride, methylsuccinic anhydride, diacetyltartaric anhydride and diglycolic anhydride. The unsaturated anhydrides are maleic anhydride, itaconic anhydride or citraconic anhydride. The resulting chitosan derivatives obtained have an acid group corresponding to the anhydride used by the formation of a covalent bond with the amino function of chitosan.

U.S. Pat. No. 4,424,346 (Hall et al.) describes chitin and chitosan derivatives useful in chelating metals, in a pharmaceutical and cosmetic formulation, chromatographic separation, enzyme immobilization, etc. These derivatives are obtained by a modification of the amine residues on the polyglucosamine to form the groups:
(a) (A)-N═CHR or —NHCH₂R
(b) —NHR′
(c) —NHR″ and
(d) —NH—CH₂COOH or —NH-glyceryl
   wherein R is an aromatic moiety having at least one hydroxyl or carboxyl group, or a macrocyclic ligand; R′ is an aldose or ketose residue and R″ is an organometallic aldehyde residue.

U.S. Pat. No. 4,659,700 (Jackson et al., 1987) discloses the preparation of a gel containing chitosan, glycerol and water for being applied to wounds.

U.S. Pat. No. 4,651,725 (Kifune et al., 1987) and 4,699,135 (Motosugi et al., 1987) claimed the preparation of chitin filaments for the manufacturing of wound dressings. Chitin is simply solubilized in dimethylacetamide in presence of lithium chloride (LiCl) at ambient temperature. This solution is extruded under pressure using a pump coagulating in a bath containing methanol. The chitin fibers are treated with a soda solution at 40% and at 80° C. for 3 hours. Then the fibers are neutralized with HCl, washed and dried.

U.S. Pat. No. 6,509,039 (Nies, 2003) discloses new chitosan derivatives by reacting pyromellitic anhydride and polymaleic anhydride having a molecular weight up to 1000. The resulting chitosan derivatives are used to produce pharmaceutical capsules, medical implants, suture materials and wound coverings.

Liu et al. [J. Mater Sci. Mater Med. (2004) vol. 15(11); pp. 1199-1203] discloses chitosan modification by coupling arginine using EDC (1-ethyl-3-(3-dimethylaminopropyl) carbodiimide) and NHS(N-hydroxysuccinimide) as coupling agents. According to the authors, the resulting polymer is a good candidate for an anticoagulant biomaterial.

U.S. Pat. No. 6,756,363 (Nordquist et al., 2004) discloses the preparation of a viscoelastic glycosylated chitosan-based solution. This chitosan derivative is obtained in the presence of a monosaccharide such as galactose, glucose and ribose via the formation of a Schiff base followed by an Amadori rearrangement. The resulting product is used in ophthalmic surgery, used as a washing solution for preventing abdominal adhesion following a surgery hemostatic dressing, as a natural polymeric substrate to separate tissues for the prevention of tendon and ligament adhesions after orthopedic surgery and for guiding tissue regeneration in dental surgery.

2.3 Problems Associated with the Modification of Biodegradable Polymers.

The problems encountered in the field of this invention are of various types.

Firstly, the modification must allow the improvement, and not the opposite, of the physical properties of the selected biodegradable polymer to be modified, such as force of traction and handiness.

Secondly, once modified, the polymers must remain biocompatible and biodegradable biomaterials. If the used polymer is a biomaterial, it must remain a biomaterial after its modification. The chemical modification reactions generally occur in an organic medium that may deteriorate or weaken the polymeric chain prematurely. In an aqueous medium, the chemical compounds used, such as EDC or NHS, can also be trapped in the material thereby making it toxic and thus not biocompatible.

Finally, the coupling agent used for modifying the polymer should not involve parasitic crosslinking reactions of polymeric chains, and such, in order to avoid biodegradable polymer denaturation and to preserve its initial properties.

There is thus a real need for a new process for the synthesis of new biocompatible, cytocompatibles and biodegradable biomaterials for using in medical, pharmaceutical or cosmetic fields, and particularly for the manufacture of dressings having hemostatic, bacteriostatic and/or wound healing properties.

### SUMMARY OF THE INVENTION

The purpose of the present invention is to satisfy the above-mentioned needs.

More precisely, a first object of the invention is a process for preparing a modified biodegradable polymer comprising:
(a) a first reactive step in aqueous medium between an amino acid, a peptide or a polypeptide and maleic anhydride to form a vinyl-carboxylic acid; and
(b) a second reactive step in aqueous medium between the vinyl-carboxylic acid obtained from step a) and a biodegradable polymer having at least a primary amine function to obtain the desired modified biodegradable polymer.

A second object of the invention is the modified biodegradable polymer obtained by the process defined above and its use for the manufacture of biomaterials having biocompatible, hemostatic and wound healing properties.

Another object of the invention is a dressing comprising the biomaterial as defined above. The hemostatic dressing according to the invention is biocompatible and has hemostatic and wound healing properties.

The present invention overcomes the difficulties encountered in the field of biomaterials and makes it possible to simply manufacture biomaterials intended to be used in the manufacture of biological dressings substitutes prosthesis and/or implants. These biomaterials have hemostatic, healing and disinfectant properties all at once, and are also biocompatible, cytocompatible and biodegradable.

The advantages of this invention are primarily due to the use, for the polymer modification process, of an aqueous medium allowing to preserve the initial properties of the polymer and to limit the presence of residual chemical compounds in the material, making the latter unsuitable to be used as a biomaterial.

Moreover, the use of an unsaturated dicarboxylic acid for modifying an aminated biodegradable polymer avoids the parasitic cross-linking reactions of these polymers.

The invention and its advantages will be better understood from the following nonrestrictive description of various preferred embodiments of the invention.

### 4. DETAILED DESCRIPTION OF THE INVENTION

As mentioned above, the present invention is directed to a process for preparing a modified biodegradable polymer. The process generally comprises a first step (a) of a reaction in aqueous medium between an amino acid, a peptide or a polypeptide and maleic anhydride (also called but-2-eneoic anhydride) to form a vinyl-carboxylic acid. In a second step (b), a reaction in aqueous medium takes place between the vinyl-carboxylic acid of step (a) and a biodegradable polymer. For second step (b) to take place, it is necessary that the biodegradable polymer used has at least one primary amino function which reacts with the double bond of the vinyl-carboxylic acid. The biodegradable polymer is obtained at the end of the second step.

### 4.1. Synthesis of the Vinyl-Carboxylic Acid Compound

From a schematic point of view, the general chemical reaction which takes place in step (a) of the process according to the invention can be represented by the following:

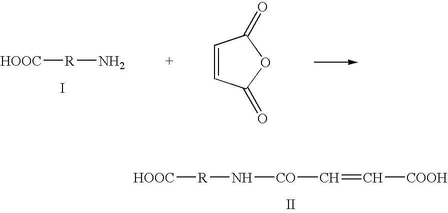

in which R represents an amino acid residue, a peptide or polypeptide residue of general formula (I) HOOC—R—NH₂.

The R residue can be selected so that the general formula (I) represents preferably an essential amino acid. More preferably, the essential amino acid is selected from glycine, L-alanine, valine, leucine, isoleucine, phenylalanine, methionine, tryptophan, serine, threonine, asparagine, glutamine, aspartic acid, glutamic acid, cysteine, tyrosin, histidine, lysine and arginine.

The R residue can also be selected so that the general formula (I) represents a nonessential amino acid. Preferably, the nonessential amino acid is selected among the residues presented in table 1 below:

TABLE 1

| | Name of the amino acid |
|---|---|
| | corresponding to formula |
| R residue value | HOOC—R—NH₂ |
| —(CH₂)₂— | β alanine |
| CH₃—CH₂—CH | α or 2-aminobutyric |
| CH₃—CH—CH₂— | β or 3-aminobutyric |
| —(CH₂)₃— | γ or 4-aminobutyric |
| CH₃—CH₂—CH₂—CH | 2-aminopentanoic or norvaline |
| CH₃—CH₂—C—CH₃ | 2-amino-2-methylbutyric or isovaline |
| —(CH₂)₄— | 5-aminopentanoic or |
| | 5-aminovaleric |
| CH₃—(CH₂)₃—CH | 2-aminohexanoic or |
| | 2-aminocaproc or |
| | norleucine |
| —(CH₂)₅— | 6-aminohexanoic |
| —(CH₂)₆— | 7-aminoheptanoic |

The R residue can be also selected so that the formula (I) represents a peptide or a polypeptide.

Finally, the R residue can also be selected so that the formula (I) represents an aromatic molecule, provided that aromatic compounds of formula (I) are water soluble.

The advantage of using the maleic acid resides in the formation of the vinyl-carboxylic acid compound of formula (II) having a double bond located in a of the dicarboxylic acid function. Consequently, the double bond is activated and will react, preferably in acidulous aqueous medium, with the amine functions of the biodegradable polymer to modify in step (b) of the process.

Moreover, the amine function will not react under these conditions with the acidic functions of the molecule of formula (II), thus avoiding any reticulation reaction between several polymeric chains. Consequently, the amino acid, peptide or polypeptide part of the molecule of formula II will remain free once grafted on the biodegradable polymer.

The reaction of step (a) is preferably carried out in aqueous medium and at a temperature ranging between about 20 and 100° C., preferably between 20 and 80° C., and more preferably between about 20 and 60° C.

The term “about” used in the present specification represents the error which can be made for the reading of a temperature, a weight, a time or a volume according to the apparatus used to carry out this measurement. The error is generally allowed as being ±10% of the read value.

One of the advantages of the process according to the present invention is the use of water as the solvent of the reaction, thus avoiding a premature destruction of the polymeric chains due to the use of an organic solvent. However, it is well-known in the art that maleic anhydride reacts in water to turn over in its hydrated state. Because of this hydrolysis reaction of anhydride in aqueous medium, the molar ratio between maleic anhydride and the aminocarboxylic acid (I) used in step (a) is between about 1/1 and 2/1, more preferably this ratio is between 1, 2/1 and 2/1, and even more preferably the molar ratio between maleic anhydride and the aminocarboxylic acid (I) is about 1.5/1.

The compound of formula (I) used in step (a) of the process according to the invention is preferably present in the reaction medium so that its concentration is very strong. More preferably the reaction medium is saturated by the compound of formula (I). According to the salvation rate of compound of formula (I), heat can be applied to facilitate its dissolution and to accelerate the coupling reaction.

Formation of the vinyl-carboxylic acid (II) (or product of coupling) is instantaneous in the form of fine powders. The product of the coupling is recovered by vacuum filtration and is washed several times with water to remove the maleic acid formed by the hydrolysis of maleic anhydride. The water for the washing is preferably cooled to about 4 to 10° C. to avoid the loss of the product from coupling. The product of the coupling is then dried. It should be noted that the obtained yield decreases when the steric hydrance of radical R increases.

4.2 Coupling Reaction of Vinyl-Carboxylic Acid with a Biodegradable Polymer

In the second step b) of the process according to the invention, the unsaturated dicarboxylic acid of general formula (II) is reacted with a natural biodegradable natural polymer having at least one amine function presents on its molecular structure.

The number of amine functions of the biodegradable polymer is variable and depends on the nature of the selected biodegradable polymer.

As aforesaid, the double bond of the vinyl-carboxylic acid, obtained from step (a) of the process, is activated by the presence of the carboxylic acid function and reacts, preferably in acidulous aqueous medium, with the amine functions of the biodegradable polymer.

According to a preferred mode of the invention, the natural biodegradable polymer used is a fibrous protein or a glycosaminoglycan.

It should be understood that from their high molecular size and the complexity of their molecular structure, the determination of the modification rate (or grafting rate) of a fibrous protein or a glycosaminoglycan is complex. In general, this modification rate can be determined by physicochemical methods such as viscosity measurements of polymer modified solutions (see the article by Durand A. et al., Biomacromolecules, 2006, vol 7 (3), p 958-64, relating to preparation of hydrophobically modified polyssaccharides. However, it is not always necessary to know this modification rate. Only the notable changes of polymer properties before and after its modification, such as the viscosity in solution, the polymer elasticity, the malleability or the like, lead to the conclusion that the polymers have been modified.

4.2.1. Modification of a Fibrous Protein. Coupling with the Vinyl-Carboxylic Acid of Formula (II)

More preferably, the fibrous protein used is collagen or elastin. Still more preferably, the fibrous protein used is collagen.

In other words, according to a preferred mode of the invention, step b) of the process comprises a covalent coupling reaction of the vinyl-carboxylic acid of formula (II) on collagen molecules.

4.2.1.1. Preparation of Collagen

The source of collagen can be prepared from tendons, ligaments, skins or all other sources containing collagen well-known in the art. The origin of collagen sources can be selected from terrestrial animals: equine, porcine, bovine, reptilic including aves; and marine animals. Placenta of human origin can be used to isolate human collagen. Any and all sources and types of collagen can be used to carry out their coupling with the vinyl-carboxylic acid of general formula (II) obtained according to step (a) of the process described hereinabove.

The collagen used can be native or crude, or be enzymatically treated by pepsin or ficin or papain to remove the telopeptide. Collagen can also be purified or pre-chemically modified.

Many methods for preparing collagen were described and are very well-known in the art. Conventional methods for the preparation of collagen: (pure, acid soluble, monomeric collagen in solution and native collagen) are incorporated herein as references, such examples of which are described in U.S. Pat. Nos. 3,934,852; 3,121,049; 3,131,130; 3,314,861; 3,530,037; 3,949,073; 4,233,360; 4,488,911; 4,216,204; 4,455,302 and 5,138,030.

According to a preferred embodiment of the invention, the collagen used in step (b) of the process is of aves origin such as chicken, goose, duck or other types of birds.

Preferably, the selected aves are chickens of about 6 to 10 weeks old from which the legs are collected. The legs are collected after slaughter and used within the next 24 hours or can be preserved at −20° C. for a later use. The legs are washed with demineralized water, then disinfected with a 70% ethanol solution, then longitudinally split along the metatarsus and fingers, and are soaked in a sterile solution of sodium chloride at a concentration ranging between 2 to 4 M (mol/L), preferably to 2 M, for 1 to 4 weeks at a temperature ranging between about 2 and 8° C. The scales surrounding the legs are removed and the tendons, ligaments and skin are removed from the bones and cut into small pieces. These materials are then put in contact in a solution of Tris(hydroxymethyl)aminomethane (usually called Tris) at 1% w/v (weight/volume)-Sodium Citrate at 2.40% (w/v), pH=7.30-7.50 for 24 hours while stirring and at a temperature ranging between about 2 and 8° C. to eliminate the remaining membranes and blood.

The materials are then collected by simple filtration and are washed with sterile water and then contacted with an acetic acid solution of 0.5 M or 3% (v/v=volume/volume) for 2 hours at ambient temperature while stirring.

By “ambient temperature” is understood a room temperature at in which the operator works and being generally between 15 and 30° C.

The materials are crushed by using a domestic blender for at least one (1) minute (min.), preferably by four (4) jolts of fifteen (15) seconds (s), or until the obtention a pasty consistency.

The collagen solution obtained is then added with a second acetic volume of acid 0.5 M (or 3% v/v) and stirred for 4 hours at ambient temperature.

A solution of pepsin (Sigma) in acetic acid 0.5 M (or 3% v/v) is added to the collagen solution for purifying collagen without telopeptides. The solution is stirred at ambient temperature for at least 24 hours, preferably between 24 to 72 hours. The solution is then centrifuged. The residue is thus drawn aside and the solution containing collagen is recovered. The solution of collagen is cooled at a temperature ranging from about 0 and 2° C. and then its pH is adjusted to about 9 with a NaOH solution (10 N and 1 N), preferably cooled at a temperature from about 0 to 2° C. to deactivate the excess pepsin. The temperature of the solution must be maintained constant at about 0 to 2° C. during the adjustment of the pH.

This solution is then centrifuged at a low temperature ranging between about 0 and 2° C. with a spinning rate of about 4200 rpm (rounds per minute) for one hour. The very viscous supernatant containing purified collagen is collected. The residue containing deactivated pepsin and telopeptides is eliminated. Purified collagen is isolated by the addition of solid NaCl while stirring to a final concentration of 2.5 M per liter of collagen solution or by addition of a 1M solution of sodium acetate. The precipitated collagen is collected by centrifugation, dissolved again in an acetic acid solution at 0.5 M (or 3% v/v) and precipitated again in a solution of NaCl (1M) or sodium acetate (1M). The collagen collected by centrifugation is washed twice with a solution of NaCl (0.3 M). Finally, purified atelopeptide collagen is dissolved in a 1% acetic acid (v/v) and is ready to be coupled to the unsaturated carboxylic acid.

The atelopeptide collagen recovered after the last washing can be dehydrated by ethanol or acetone, dried and preserved for later use.

4.2.1.2. Coupling Reaction Between Collagen and the Vinyl-Carboxylic Acid of Formula (II)

Collagen, such as the atelopeptide collagen obtained according to the process described hereinabove, is dissolved in an acetic acid solution of about 1% (v/v).

The vinyl-carboxylic acid of general formula (II) (also called “coupling agent” hereinafter) is added into the collagen solution while stirring for one hour. The solution becomes viscous and is heated to 37° C. for 15 minutes or until the dissolution of the coupling agent is complete. The coupled collagen solution becomes more fluid and the stirring is maintained for about 1 to 4 hours at about 20° C.

Thereafter, the coupled collagen is precipitated by adding the equivalent 1M solid NaCl or 1M of sodium acetate per liter of collagen solution. The precipitate is recovered by centrifugation for 45 minutes at a spinning rate of about 4200 rpm and at a temperature from about 2 to 4° C. Coupled collagen is dissolved again in a 1% (w/v) acetic acid solution, and collagen is precipitated for a second time by adding a solution of 0.8 M of NaCl or 1M of sodium acetate while stirring. Stirring is maintained for about one to 4 hours to complete the precipitation.

The resulting new material is recovered by centrifugation or filtration and is then washed by a solution of Tris(hydroxymethyl)aminomethane 1% (w/v), with pH=7.00±0.30. Finally the biomaterial, collected by centrifugation, is dehydrated using pure ethanol and dried at 20-25° C. under a laminar flow hood.

4.2.2. Modification of a Glycosaminoglycan with the Vinyl-Carboxylic Acid of Formula (II)

As mentioned hereinabove, according to a preferred embodiment of the invention, the biodegradable natural polymer used in step b) of the process according to the invention is a glycosaminoglycan.

More preferably, the glycosaminoglycan used is chitosan extracted from chitin.

The chitosan used must contain at least 75% of amino groups (NH₂—), which corresponds to deacetylated chitin having a degree of deacetylation of at least 75%.

According to a more preferred embodiment of the invention, the chitosan used is commercial chitosan extracted from crab shells with a degree of deacetylation higher than 85% (Chitosan referred to as C3646 at Sigma).

The chitosan is solubilized in acetic acid at 3% w/v. The vinyl-carboxylic acid is added in the form of powder under strong stirring conditions. The mixture is heated to about 60° C. and this temperature is maintained until the total dissolution of the acid. The heating is then stopped and stirring is kept for about two (2) hours.

The coupled chitosan is precipitated by addition of a solution of NaCl (1M) or sodium acetate (1M) per liter of chitosan solution. The precipitate is recovered by centrifugation or filtration and is washed, at least twice, with pure water.

The washed precipitate is finally recovered by centrifugation or filtration and is dehydrated with ethanol or acetone and air dried at about 20-25° C.

Another object of the present invention is the modified biodegradable polymer obtained by the process described hereinabove, as well as the use of this polymer in the manufacture of biomaterials.

The biomaterials obtained are particularly used in the medical field, and more particularly in surgery, pharmacy, dermatology, esthetics and cosmetics. The biomaterials according to present invention are biocompatible. Indeed, the presence of the amino acids, peptides or polypeptides grafted along the polymeric chain accentuates the properties of biocompatibility. The biomaterial will be thus tolerated by tissues, which allows for the increase of the hemostatic and wound healing properties of the original biodegradable polymer.

Among these products, a preferred embodiment of the invention relates to the use of the modified polymer according to the invention for the manufacture of a dressing with hemostatic and wound healing properties.

The dressing according to the invention can be manufactured in the form of powder, sponges, gels, films or creams.

The sponge can be manufactured by the freeze-drying process known in the art.

It has to be understood that the dressing according to the invention may also contain a certain amount of biodegradable unmodified polymers used in step (b) of the process according to the invention and that has not reacted.

The transparent film can be manufactured by drying under ventilation at a temperature ranging between about 20 and 25° C. The time of drying mentioned above varies according to the volume and thickness of the film or sponge.

The powder, the sponge, the film, the cream or the gel can be individually packed in polypropylene bottles or aluminum bags, and are ready to be sterilized by gamma-rays, a sterilization technique also well-known in the art.

Pharmaceutically acceptable ingredients which are soluble in the same medium as collagen, such as an antibiotic, an antiseptic, an anticancer or mixture thereof, are incorporated before filtration and freeze-drying.

The biodegradable polymer, by virtue of its structural modification following the coupling with the vinyl-carboxylic acid of formula (II), influences the structure of the network made from the polymeric chains of the manufactured material.

Indeed, the resulting polymer network is denser. Therefore, the crushing (or collapsing) phenomenon of meshes during the drying of the films prepared with this compound can be avoided, translated by a maintenance of the mesh size (without shrinking) for air drying or cold and vacuum (freeze-drying) drying and a notable increase in the following properties:
liquid absorption of with a three-dimensional expansion of the product (surface and thickness);
the elasticity of the polymer (force of traction increased);
adherence to the tissue to be treated;
malleability;
handiness; and
viscosity in solution.

Among other possible applications for the polymer according to the invention, there are products prepared for a use in medicine such as artificial tissues or replacement organs such as skin (in the treatment of burns), bones (prosthesis and implants), ligaments or tendons (implants).

Hemostatic dressings according to the invention allow faster wound healing, making it thus possible to reduce residual scars.

The biomaterials according to the invention can be used in esthetics and cosmetics, to fight against skin aging (wrinkles) as well as residual scars resulting from accidents, acne or burns.

It has to be understood that the biomaterials according to the invention, and particularly those in the form of powder, can be encapsulated in the form of micro-beads, micro-capsules or implants for a controlled out in vivo salting.

### 5. EXAMPLES

The examples detailed hereinbelow describe various syntheses of vinyl-carboxylic acid compounds and their uses in processes for preparing new biomaterials containing collagen or chitosan.

### 5.1. Preparation of HOOC—CH₂—NH—CO—CH═CH—COOH

75 g (1 mole) of glycine (HOOC—CH₂—NH₂) is dissolved in 0.3 liter of demineralized water. 147 g (1.5 moles) of powdered maleic anhydride is added to the solution of glycine under strong stirring conditions all at once. White and very thin precipitates appear in the reactional medium as soon as the anhydride dissolves. Stirring is maintained for about 30 more minutes and the resulting coupled product is filtered under a vacuum, abundantly washed with demineralized water cooled to 2-4° C., until the washing water becomes slightly acid (pH≦4 to 5). The pH is controlled with pH indicator paper. After washing, the resulting product is air dried at ambient temperature.

147 g of dry product are obtained, which is equivalent to a yield of about 85%.

Proton nuclear magnetic resonance spectra (NMR-¹H) were carried out on a Brucker AMX-400® operating at 400 MHz.

NMR-¹H (400 MHz, DMSO): δ=3.90 ppm (d, 1H); 6=6.30 ppm (d, 1H); δ=6.41 ppm (d, 1H); δ=9.18 ppm (m, 1H).

### 5.2. Preparation of the Vinyl-Carboxylic Acid of Formula HOOC—CH₂—CH₂—NH—CO—CH═CH—COOH

89.09 g (1 mole) of (1 mole) of β-alanine (HOOC—CH₂—CH₂—NH₂) dissolved in 0.3 L of demineralized water. 147 g (1.5 mole) of powdered maleic anhydride are added to the solution under strong stirring conditions all at once. White and very thin precipitates appear as soon as the anhydride dissolves into the reactional medium. Stirring is maintained for about 30 more minutes and the resulting product is filtered under a vacuum, abundantly washed with demineralized water until the washing water becomes slightly acidic (pH≈4 to 5). The pH is controlled with pH indicator paper. After washing, the coupled product is air dried at ambient temperature.

140 g of dry product are obtained with a yield of about 75%.

### 5.3 Preparation of the Vinyl-Carboxylic Acid of Formula HOOC—(CH₂)₅—NH—CO—CH═CH—COOH

131 g (1 mole) of 6-aminohexanoïque acid (C₆H₁₃O₂N) are dissolved into 0.5 L of demineralized water. 147 g (1.5 mole) of powdered maleic anhydride are added to the 6-aminohexanoïque acid solution under strong stirring conditions all at once. White and very thin precipitates appear as soon as the anhydride dissolves into the reactional medium. Stirring is maintained for about 30 more minutes and the resulting product is filtered under a vacuum, abundantly washed with demineralized water until the washing water becomes slightly acidic (pH≈4 to 5). The pH is controlled with pH indicator paper. After washing, the coupled product is air dried at ambient temperature.

164 g of dry product (C₁₀H₁₅O₅N) are obtained with a yield of about 71.6%.

NMR-¹H (400 MHz, DMSO): δ=1.30 ppm (m, 2H); δ=1.48 ppm (m, 4H); δ=2.2 ppm (t, 2H); δ=3.15 ppm (m, 2H); δ=6.26 ppm (d, 1H); δ=6.40 ppm (d, 1H); δ=9.10 ppm (s, 1H).

### 5.4 Preparation of Atelopeptide Collagen

The legs of 8 week old chickens are collected 2 hours after slaughter. These legs are brushed, washed and rinsed with demineralized water, then disinfected by soaking in a 70% ethanol solution for about one hour. The legs are longitudinally split on the level of the metatarsus and fingers before soaking them in a sterile solution of sodium chloride 2 M (mol/L), for 2 weeks at a temperature ranging from 2 to 8° C. The NaCl solution (2 M is changed twice a week. The scales surrounding the legs are removed and the tendons, ligaments and skin are removed from the bones and then cut into small pieces which are then put into a solution of Tris(hydroxymethyl)aminomethane (Tris) 1% (w/v)-sodium 2.40% Citrate (w/v), pH=7.30-7.50 under stirring conditions for 24 hours and at 2-8° C. to eliminate the membrane remains and the blood. The materials are recovered by simple filtration and are washed with sterile water.

75 g of tendons, ligaments and skins are brought into contact under stirring conditions in 1 L of acetic acid (0.5 M or 3% (v/v)) for 2 hours at ambient temperature, and are then crushed by using a domestic blender. The whole crushing process lasts for 1 minute (4×15 seconds) or until a pasty consistency is obtained. The collagen solution is added with a second volume (1 L) of acetic acid (0.5 M or 3%) and stirring is maintained for 4 hours at ambient temperature. 2.25 g of pepsin of the mucosa of porcine origin (Sigma), that is to say 3% in weight compared to the chicken pieces (according to the U.S. Pat. No. 6,448,378), are dissolved into 10 mL of acetic acid (0.5 M). This solution is added to the solution of chicken pieces. Stirring is maintained for 64 hours at ambient temperature. The solution is then centrifuged at a spinning speed of 4200 rpm for 1 hour at 2-4° C. The supernatant containing collagen is recovered and cooled at 0-2° C. The pH of the collagen solution is adjusted at pH=9.0+−0.10 with a cooled solution at about 0-2° C. of NaOH 10N and 1 N when the pH of the solution reaches 8.0; in order to denature pepsin. The temperature is kept at about 0-2° C. for the pH adjustment (U.S. Pat. No. 5,874,537). The rest of the solution is kept at about 0-2° C. for 16 hours and it is then centrifuged at 4200 rpm (Beckman J6™) for 1 hour and at 2-4° C. Two parts of the solution are separated, the clear and very viscous part containing atelopeptide collagen is transferred into a beaker and is kept at 0-2° C., the increase of the temperature beyond 6° C. involving the formation of collagen gels. The residue containing pepsin and the telopeptides is eliminated. The resulting collagen solution may be directly reacted with selected vinyl-carboxylic acid for coupling.

The equivalent of 2.5 M of solid NaCl (or 1 M of sodium acetate) is added under stirring conditions into the collagen solution cooled to about 0-2° C. Collagen is instantaneously precipitated; stirring is maintained for 2 to 4 hours and the solution is left to settle for an entire night at ambient temperature. Collagen is recovered by centrifugation at 4200 rpm for 1 hour and is solubilized again in a 3% acetic acid solution at about 0-2° C. Collagen is purified again by a second precipitation by adding 1M solid NaCl (or 1M of sodium acetate) into the solution under stirring conditions. Precipitated collagen is recovered by centrifugation at 4200 rpm for 1 hour and 0-2° C. The precipitate is then washed with a solution of Tris(hydroxymethyl)aminomethane 1% (w/v) with pH=7-7.50. The washing solution is eliminated by centrifugation at 4200 rpm for 1 hour and at 0-2° C., and the precipitate is dehydrated using pure ethanol and dried at 20-25° C. 9.40 g of collagen are obtained.

### 5.5 Preparation of Coupled Collagen

### 5.5.1. Direct Coupling of Atelopeptide Collagen

Two (2) liters of atelopeptide collagen solution prepared as previously described (before lyophilization) are cooled at 0-2° C., pH=9.0 (±0.10). 10 g of vinyl-carboxylic acid of chemical formula HOOC—CH₂—NH—CO—CH═CH—COOH as previously prepared is added to the collagen solution, under stirring conditions for at least 1 hour. The solution is heated to 37° C. for at least 30 minutes or until the total dissolution of the acid. The temperature is left to lower to 20° C. under stirring conditions for an additional period of time, from 2 to 4 hours. Coupled collagen is precipitated by adding to the solution, and under stirring conditions, the equivalent of 2.5M of solid NaCl or 1M of sodium acetate. A thin precipitate appears. Stirring is kept for 1 hour and left to settle at ambient temperature for an entire night. Coupled collagen is recovered by centrifugation at 4200 rpm for 1 hour and 0-2° C. The precipitate is then dissolved again into an equal volume before the first precipitation with an acetic acid solution at 1% (v/v). The second precipitation of coupled collagen was made by adding to the solution, and under stirring conditions, an equivalence of 0.8M of solid NaCl or 1M of sodium acetate. Coupled collagen is purified again and is again recovered by centrifugation at 4200 rpm for 1 hour and about 0-2° C. Coupled collagen is then washed with a solution of Tris(hydroxymethyl)aminomethane 1% (w/v), pH=7.0-7.5. The washing solution is eliminated by centrifugation at 4200 rpm for 1 hour and 0-2° C. and the precipitate is dehydrated using pure ethanol and dried at 20-25° C. 8.20 g of modified collagen are obtained.

### 5.5.2. Coupling of Purified Atelopeptide Collagen

10 g of dehydrated atelopeptide collagen obtained according to the process described hereinabove is finely ground and dissolved into 500 mL of acetic acid 1% (v/v). The final concentration of collagen is 2% (w/v).

2.5 g of unsaturated vinyl-carboxylic acid of formula HOOC—CH₂—NH—CO—CH═CH—COOH are added to the collagen solution under stirring conditions for 1 hour at ambient temperature. The collagen solution becomes more viscous. The temperature of the temperature is raised to 37° C. during at least 30 minutes or until the complete dissolution of the coupling agent. Coupled collagen is precipitated by adding an equivalence of 1M of solid NaCl or 1M of sodium acetate to the solution. Stirring is maintained for at least 1 hour or overnight at ambient temperature. The precipitate is recovered by centrifugation at 4200 rpm, for 1 hour and at 0-2° C. (Beckman J6™). The precipitate is then dissolved again into a volume equal to the one before the first precipitation with an acetic acid solution at 1% (v/v). The second precipitation of coupled collagen was performed by adding to the solution under stirring conditions an equivalence of 0.8M of solid NaCl or 1M of sodium acetate. Coupled collagen is purified again and collected by centrifugation at 4200 rpm for 1 hour and 0-2° C. Coupled collagen is then washed with a solution of Tris(hydroxymethyl)aminomethane 1% (w/v), pH=7.0-7.5. The washing solution is eliminated by centrifugation at 4200 rpm for 1 hour at 0-2° C. and the precipitate is dehydrated using pure ethanol and dried at 20-25° C. 7.02 g of modified collagen are obtained.

### 5.6 Preparation of the Coupled Chitosan

The second compound used in this invention is commercial chitosan from crab shells, and 85% deacetylated (C3646 Sigma).

10 g of chitosan (1%) are dissolved in 1 L of acetic acid solution at 0.5 M or 3%. The pH of the solution is 3.40. The solution is filtered to eliminate debris and impurities. 10 g of about vinyl-carboxylic acid of formula:

HOOC—CH₂—NH—CO—CH═CH—COOH

is added to the chitosan solution under stirring conditions at ambient temperature, the pH of the mixture being of about 3.50. The mixture is heated at 55-60° C. for at least 1 hour or until the total dissolution of the coupling agent. The temperature is lowered to 20° C. under stirring conditions, and then the coupled chitosan is precipitated by adding 1M of solid NaCl or 1M of sodium acetate into the solution under stirring for at least 1 hour. The mixture is centrifuged at 4200 rpm for 1 hour with 0-2° C. (Beckman J6™) to recover the coupled chitosan. The precipitate is washed twice with pure water and finally the coupled chitosan is collected by centrifugation at 4200 rpm for 1 hour at about 0-2° C. The chitosan is dehydrated with ethanol and dried at ambient temperature. 9.55 g of chitosan are obtained.

### 5.7 Preparation of Biomaterials and Hemostatic Dressings

### 5.7.1. Preparation of a Biomaterial Containing Modified Collagen or Chitosan

1 g of coupled collagen or coupled chitosan obtained according to the examples hereinabove is dissolved into 100 mL of an acetic acid solution at 1% (v/v). 1 mL of a solution at 10% (v/v) of polyoxyethylene sorbitan monooleate (Tween™ 80) is added. The bubbles formed due to the viscosity of the solution are eliminated using ultrasound or a vacuum.

The solution thus prepared is a biomaterial which can be used either directly in the medical, biomedical, pharmaceutical or cosmetic field, or for the manufacture of dressings.

Acceptable pharmaceutical ingredients such as antibiotics, bactericides, or anti-cancer ingredients can be incorporated into this biomaterial in liquid form before making the dressing.

As aforesaid, the dressing can be prepared in different forms such as a sponge, a film, a powder, a gel or a cream. The form will be selected according to the desired use for this dressing.

### 5.7.2 Preparation of Hemostatic Sponges

The solution prepared above is poured into anti-adhesive moulds. The volume of the solution is given according to the dimension of the mould and the thickness of the dressing. The moulds are placed on the shelves of a freeze drier at 20° C. (FTS System) programmed to reduce the temperature from the shelves to 0° C., at a rate of 1° C. per minute. The temperature is maintained at 0° C. for 1 hour, then the temperature continues to lower to −20° C. and is maintained for 1 hour. After this period of time, the temperature lowers to −40° C. and is maintained for 1 hour so that ice formation be quite homogeneous without the formation of crystals on the surface. A vacuum of 200 mtorr is then applied and at the same time the temperature of the shelves increases to 20° C. at a rate of 0.02° C. per minute. The vacuum is lowered to 20 mtorr and the temperature continues to increase to 30° C. and is still maintained for 2 hours before the end of the freeze-drying cycle. Freeze-dried sponges or dressings are removed from their mould and individually packaged in aluminum bags. These bags are sterilized using gamma-rays.

### 5.7.3 Preparation of Transparent Films

The solution prepared above is poured into moulds made of polycrystal, polycarbonate or polystyrene. The volume of the solution is selected according to the dimensions of the mould and film thickness. The moulds are then placed under a laminar flow hood. The drying lasts about 48 to 60 hours at ambient temperature. The films are separated from their mould and are individually packaged in aluminum bags. These bags are sterilized using gamma-rays.

### 5.7.4 Preparation of Hemostatic Powders

Coupled collagen or coupled chitosan obtained according to the process disclosed hereinabove are ground to obtain a very fine powder. This powder is packaged in polypropylene bottles closed by a stopper. These bottles are sterilized using gamma-rays.

Pharmaceutically acceptable ingredients such as antibiotics, bactericides or anti-cancer may be incorporated into the powder.

### 5.8 Tests for the Hemostatic Effect of Sponges Containing Modified Collagen and Modified Chitosan

The tests for the hemostatic effect of the dressings obtained in the form of sponges were performed on rabbit livers. Tested dressings are prepared with modified collagen and modified chitosan according to the process described hereinabove. Each dressing has a surface of about 5 cm by 3 cm and a thickness of about 0.4 cm. The placebo used is a compress folded into four layers to obtain a thickness equivalent to that of hemostatic sponges.

The rabbits are anaesthetized and incised longitudinally in the abdomen; the exposed liver is cut at the end of the lobe into a piece of about 2 cm×0.5 cm. Sponges are applied directly to the wounds while holding with the hand without applying a strong pressure. A stronger manual pressure is applied with the compress. The hemostasis is observed every 30 seconds (s) until the bleeding completely stops.

Table 2 below summarizes the stopping of the bleeding.

TABLE 2

| | coupled Collagen | Coupled Chitosan | Compress |
|---|---|---|---|
| Rabbit | 1 | 2 | 3 |
| Stop time | 90 s | 110 s | 250 s |

The hemostatic sponges have been also applied to accidental cuts of the human skin. The application of sponge caused a quick stop (but not quantified) of the bleeding and an accelerated wound healing with the absence of residual scars.

Although preferred embodiments of the invention have been described in detail above, the invention is not only limited to these preferred embodiments and several changes and modifications can be made by a person of the art without departing from the nature and spirit of the invention.

1. Process for preparing a modified biodegradable polymer comprising:
(a) a first reactive step in aqueous medium between an amino acid, a peptide or a polypeptide and the maleic anhydride to form a vinyl-carboxylic acid; and
(b) a second reactive step in acidulous aqueous medium between the vinyl-carboxylic acid obtained from step a) and a biodegradable polymer having at least a primary amine function to obtain the desired modified biodegradable polymer, the biodegradable polymer being selected from a glycosaminoglycan or a fibrous protein.

2. The process according to claim 1, wherein the amino acid is an essential amino acid selected from glycine, L-alanine, valine, leucine, isoleucine, phenylalanine, methionine, tryptophane, serine, threonine, asparagine, glutamine, aspartic acid, glutamic acid, cysteine, tyrosine, histidine, lysine and arginine, or a non essential amino acid selected from β-alanine, 2-aminobutyric acid, 3-aminobutyric acid, 4-aminobutyric acid, 2-aminopentanoïc acid, 2-amino-2-methylbutyric acid, 5-aminopentanoïc acid, 6-aminohexanoïc acid and 7-aminoheptanoïc acid.

3. The process according to claim 1, wherein the aqueous medium used in step a) is pure demineralized water.

4. (canceled)

5. The process according to claim 1, wherein the glycosaminoglycan is chitosan.

6. The process according to claim 5, wherein the chitosan has a degree of deacetylation superior to about 75%.

7. The process according to claim 6, wherein the chitosan has a degree of deacetylation superior or equal to about 85%.

8. The process according to claim 1, wherein the acidulous aqueous medium used in the step b) is an acetic acid solution of concentration of between about 1% to about 3% in volume of acetic acid by volume of solution.

9. The process according to claim 1, wherein the fibrous protein is collagen or elastin.

10. The process according to claim 9, wherein the collagen is native collagen or an atelopeptide collagen.

11. A modified biodegradable polymer obtained by the process according to claim 1, said polymer having biocompatible, hemostatic and healing properties.

12. A biomaterial comprising a modified biodegradable polymer obtained by the process according to claim 1 said biomaterial having medical, biomedical, pharmaceutical or cosmetic applications.

13. The biomaterial according to claim 12, wherein the biomaterial further comprises a biodegradable polymer identical to the one used in step b) of the process according to claim 1, an excipient pharmaceutically acceptable and/or an ingredient pharmaceutically acceptable selected from antibiotics, antiseptics, anticancer and mixtures thereof.

14. The biomaterial according to claim 12, wherein the biomaterial is in solid form or in aqueous solution.

15. A dressing comprising a biomaterial as defined in claim 12, said dressing being biocompatible and having hemostatic and healing properties.

16. The dressing according to claim 16, in the form of a sponge, a powder, a film, a gel or a cream.
